(19) United States
(12) Patent Application Publication
(10) Pub. No.: US 2024/0138811 A1
(43) Pub. Date: May 2, 2024

(54) CASING FOR A PORTABLE ULTRASONIC IMAGING DEVICE

(71) Applicant: Exo Imaging, Inc., Santa Clara, CA (US)

(72) Inventors: Sandeep Akkaraju, Wellesley, MA (US); Steven Bowers, Oak Park, CA (US)

(73) Assignee: Exo Imaging, Inc., Santa Clara, CA (US)

(21) Appl. No.: 18/548,835

(22) PCT Filed: Mar. 31, 2021
(86) PCT No.: PCT/US2021/025198

§ 371 Date: Sep. 1, 2023

(51) Int. Cl. A61B 8/00; A61B 8/08

(52) U.S. Cl. CPC A61B 8/4427; A61B 8/4422; A61B 8/4433; A61B 8/5207; A61B 8/565

(57) ABSTRACT

A casing to store a portable imaging device, a method to be performed at the casing, and a machine-readable medium to cause one or more processors to implement the method. The imaging device is adapted to generate imaging data corresponding to a target being imaged using ultrasonic energy. The casing adapted to be opened and closed, and includes an exterior housing; an interior portion within the exterior housing to house the imaging device therein; a memory; a one or more processors coupled to the memory to perform computations on the imaging data from the imaging device to at least one of cause an image of the target to be displayed on a display or cause the imaging data to be stored in the memory; and a power source to supply charge to the imaging device.

### FIELD

Embodiments relate in general to casings, and in particular to casings for imaging devices, such as portable ultrasonic imaging devices or probes including those with micromachined ultrasound transducers (MUTs).

### BACKGROUND

Micromachined ultrasound transducer (MUT) technology has enabled ultrasound or ultrasonic imaging probes or imaging devices that are smaller in overall size and weight and have lower operating power requirements compared to more conventional ultrasound imaging technology. Some MUT-based ultrasonic imaging devices, including those that are based on capacitive MUT (cMUT) or piezoelectric MUT (pMUT) technologies, are handheld devices that are powered with a battery power source. A smaller overall form factor and the optional battery source for power have allowed ultrasonic imaging devices to become portable, a feature that was not previously possible with prior generation ultrasonic imaging devices.

The portable nature of MUT-based ultrasonic imaging devices raises a new set of functionality and operability benefits and challenges. For example, a portable ultrasonic imaging device may, by virtue of its portability, be subjected to environmental and/or mechanical stresses not experienced by non-portable ultrasonic imaging devices, the operational environment of which is typically well controlled. For example, MUT-based ultrasonic imaging devices are more likely to be dropped/subjected to structural shock, to be used in outdoor conditions where they may be exposed to moisture, direct sunlight, or extreme temperatures. In addition, MUT-based ultrasonic imaging devices are more likely to be used in locations where external power sources are scarce or not readily available.

Although the portability and lower operating power requirements of the MUT-based ultrasonic imaging device means that it can be supplied with power by way of a battery, batteries nevertheless provide a limited duration source of power. Although a MUT-based ultrasonic imaging device may operate using disposable batteries, an alternative solution is to have the probe operate using rechargeable battery cells or battery packs, such as those using lithium-ion technology. Similarly, however, the available power from a rechargeable battery will also eventually drain and reach a threshold under which the imaging device is no longer operable and the battery requires a recharge. Typically, rechargeable batteries are recharged through use of dedicated charging stations plugged into an electrical outlet, such as an alternating current (AC) outlet, for example a wall-socket. In remote deployment conditions where a MUT-base probe is being used, however, such a source of battery recharge may not be readily available, accessible or convenient.

### BRIEF DESCRIPTION OF THE DRAWINGS

Some of the features of the embodiments are set forth with particularity in the appended claims. A better understanding of the features and advantages of embodiments will be obtained by reference to the following detailed description, in which the principles of the embodiments are utilized, and the accompanying drawings (also “Figure” and “Fig.” herein), of which:

FIG. 1 is a block diagram of an imaging device in accordance with some embodiments.

FIG. 2 is a schematic diagram of an imaging system in accordance with some embodiments.

FIG. 3 is a schematic diagram of an imaging device in accordance with some embodiments.

FIG. 4 is a top plan view of a base portion of a casing according to some embodiments.

FIG. 5 is a top plan view of a casing according to one embodiment.

FIG. 6 is a top plan view of a casing according to a further embodiment.

FIG. 7 is a schematic diagram of a wireless communication circuitry of an embodiment of a casing, and of wireless networks within which the wireless communication circuitry may cause wireless communication.

FIG. 8 is a flow diagram of a method according to an embodiment.

### DETAILED DESCRIPTION

Some embodiments provide a casing to store a portable imaging device adapted to generate imaging data corresponding to a target being imaged using ultrasonic energy, the casing adapted to be opened and closed, and including: an exterior housing; an interior portion within the exterior housing to house the imaging device therein; a memory; a one or more processors coupled to the memory to perform computations on the imaging data from the imaging device to at least one of cause an image of the target to be displayed on a display or cause the imaging data to be stored in the memory; and a power source to supply charge to the imaging device.

Advantageously, some embodiments provide a casing for an ultrasonic imaging device that is configured to protect the casing from mechanical impact or shock while providing an auxiliary power source to charge the imaging device, and while optionally providing wired or wireless communication to one or more processors within the casing to allow a communication of imaging data between the casing and the imaging device, an external display, or a remote device (i.e. a device that is not at the same location as the casing and the imaging device). Further advantages of embodiments will become apparent at the description progresses.

In general, the embodiments relate to casings for imaging devices, and more particularly to casings for imaging devices having ultrasonic transducer elements.

An “ultrasonic waveform” as mentioned herein, for example in a medium such as water, flesh, lens, etc., may, in some embodiments, refers to a compensation of the waveforms of each of the transmitting transducer elements. Although the transducer elements, such as groups of transducer elements, according to some embodiments, may sometimes fire together, they may often be fired separately from one another (e.g. to steer).

Some embodiments of an imaging device may additionally include hardware and/or software to receive reflected ultrasonic energy from an object to be imaged, and to convert the received ultrasonic energy into electrical signals.

Some embodiments of an imaging device may further include hardware and/or software to construct an image of the object to be imaged, to cause a display of the image, and/or to display the image.

To perform the imaging, an imaging device may transmit an ultrasonic waveform into body tissue toward an object to be imaged, and receive reflected ultrasonic energy from the object. Such an imaging device may include one or more transducer elements, and which may function using photo-acoustic or ultrasonic effects. Such transducer elements may be used for imaging, and may further be used in other applications. For example, the transducer elements may be used in medical imaging, for flow measurements in pipes, in speaker and microphone arrays, in lithotripsy, for localized tissue heating for therapeutic purposes, and in highly intensive focused ultrasound (HIFU) surgery.

In the context of embodiments, although ultrasonic waveforms, ultrasonic waves, ultrasonic pressure waves, and/or the use of ultrasound is called out expressly, embodiments are not limited to ultrasound specifically, and include within their scope the generation and processing of waves that can propagate in a body, be reflected back from an object of the body, and be decoded/analyzed/processed to allow generation of information pertaining to the object, such as the generation of an image corresponding to the object on a display device.

Traditionally, imaging devices such as ultrasound imagers used in medical imaging use piezoelectric (PZT) materials or other piezo ceramic and polymer composites. Such imaging devices may include a housing to house the transducers with the PZT material, as well as other electronics that form and display the image on a display unit. To fabricate the bulk PZT elements or the transducers, a thick piezoelectric material slab can be cut into large rectangular shaped PZT elements. These rectangular-shaped PZT elements can be expensive to build, since the manufacturing process involves precisely cutting generally the rectangular-shaped thick PZT or ceramic material and mounting it on substrates with precise spacing. Further, the impedance of the transducers is much higher than the impedance of the transmit/receive electronics for the transducers, which can affect performance.

Embodiments of the present disclosure may be utilized in the context of imaging devices that utilize either piezoelectric micromachined ultrasound transducer (pMUT) or capacitive micromachine ultrasonic transducer (cMUT) technologies, as described in further detail herein.

In general, MUTs, such as both cMUT and pMUT, include a diaphragm (a thin membrane attached at its edges, or at some point in the interior of the probe), whereas a “traditional,” bulk PZT element typically consists of a solid piece of material.

Piezoelectric micromachined ultrasound transducers (pMUTs) can be efficiently formed on a substrate leveraging various semiconductor wafer manufacturing operations. Semiconductor wafers may currently come in 6 inch, 8 inch, and 12 inch sizes and are capable of housing hundreds of transducer arrays. These semiconductor wafers start as a silicon substrate on which various processing operations are performed. An example of such an operation is the formation of SiO 2 layers, also known as insulating oxides. Various other operations such as the addition of metal layers to serve as interconnects and bond pads are performed to allow connection to other electronics. Yet another example of a machine operation is the etching of cavities. Compared to the conventional transducers having bulky piezoelectric material, pMUT elements built on semiconductor substrates are less bulky, are cheaper to manufacture, and have simpler and higher performance interconnection between electronics and transducers. As such, they provide greater flexibility in the operational frequency of the imaging device using the same, and potential to generate higher quality images.

In some embodiments, the imaging device may include an application specific integrated circuit (ASIC) that includes one or more transmit drivers, sensing circuitry to process electrical energy corresponding to received ultrasound energy reflected back from the object to be imaged (echo signals), and other processing circuitry to control various other operations. The ASIC can be formed on another semiconductor wafer, or on the same semiconductor wafer. This ASIC can be placed in close proximity to pMUT elements to reduce parasitic losses. As a specific example, the ASIC may be 50 micrometers (μm) or less away from a transducer array including the pMUT elements. In a broader example, there may be less than 100 μm separation between the 2 wafers or 2 die, where each wafer includes many die and a die includes a transducer in the transducer wafer and an ASIC in the ASIC wafer. In some embodiments, the ASIC has a matching footprint relative to the pMUT transducer that includes the pMUT elements, and thus may be stacked for wafer-to-wafer interconnection with the pMUT transducer die, for example with an ASIC wafer being stacked with the transducer die or an ASIC die itself being stacked with the transducer die through interconnects. Alternatively, the transducer can also be developed on top of the ASIC wafer as a single device using low temperature piezo material sputtering and other low temperature processing compatible with ASIC processing.

Wherever the ASIC and the transducer interconnect, according to one embodiment, the two may have similar footprints. More specifically, according to the latter embodiment, a footprint of the ASIC may be an integer multiple or divisor of the pMUT footprint.

Regardless of whether the imaging device uses pMUT elements or cMUT elements in its transducer(s), an imaging device according to some embodiments may include a number of transmit channels and a number of receive channels. Transmit channels are to drive the transducer elements with a voltage pulse at a frequency the elements are responsive to. This causes an ultrasonic waveform to be emitted from the elements, which waveform is to be directed towards an object to be imaged, such as toward an organ in a body. In some examples, the imaging device with the array of transducer elements may make mechanical contact with the body using a gel in between the imaging device and the body. The ultrasonic waveform travels towards the object, i.e., an organ, and a portion of the waveform is reflected back to the transducer elements in the form of received/reflected ultrasonic energy where the received ultrasonic energy may converted to an electrical energy within the imaging device. The received ultrasonic energy may then be further processed by a number of receive channels to convert the received ultrasonic energy to electrical signals, and the electrical signals may be processed by other circuitry to develop an image of the object for display based on the electrical signals.

An embodiment of an ultrasonic imaging device includes a transducer array, and control circuitry including, for example, an application-specific integrated circuit (ASIC), and transmit and receive beamforming circuitry, and optionally additional control electronics. Each of the number of transmit and/or receive channels may be dynamically controlled, for example by control circuitry of the image device, to reduce power, or may be powered down entirely. Additionally, other characteristics of each channel may also be configurable.

In an embodiment, an imaging device may include a handheld body or housing where transducers and associated electronic circuitries, such as control circuitry and optionally a computing device are housed. The imaging device may also contain a battery to power the electronic circuitries.

Thus, some embodiments pertain to a portable imaging device utilizing either pMUT elements or cMUT elements in a 2D array. In some embodiments, such an array of transducer elements is coupled to an application specific integrated circuit (ASIC) of the imaging device.

In the following description, for purposes of explanation, specific details are set forth in order to provide an understanding of the disclosure. It will be apparent, however, to one skilled in the art that the disclosure can be practiced without these details. Furthermore, one skilled in the art will recognize that examples of the present disclosure, described below, may be implemented in a variety of ways, such as a process, one or more processors (processing circuitry) of a control circuitry, one or more processors (or processing circuitry) of a computing device, a system, a device, or a method on a tangible computer-readable medium.

One skilled in the art shall recognize: (1) that certain fabrication operations may optionally be performed; (2) that operations may not be limited to the specific order set forth herein; and (3) that certain operations may be performed in different orders, including being done contemporaneously.

Elements/components shown in diagrams are illustrative of exemplary embodiments and are meant to avoid obscuring the disclosure. Reference in the specification to “one example,” “preferred example,” “an example,” “examples,” “an embodiment,” “some embodiments,” or “embodiments” means that a particular feature, structure, characteristic, or function described in connection with the example is included in at least one example of the disclosure and may be in more than one example. The appearances of the phrases “in one example,” “in an example,” “in examples,” “in an embodiment,” “in some embodiments,” or “in embodiments” in various places in the specification are not necessarily all referring to the same example or examples. The terms “include,” “including,” “comprise,” and “comprising” shall be understood to be open terms and any lists that follow are examples and not meant to be limited to the listed items. Any headings used herein are for organizational purposes only and shall not be used to limit the scope of the description or the claims. Furthermore, the use of certain terms in various places in the specification is for illustration and should not be construed as limiting.

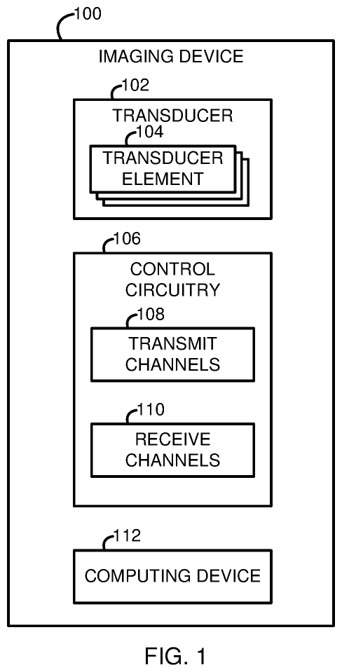

Turning now to the figures, FIG. 1 is a block diagram of an imaging device 100 with a controller or control circuitry 106 controlling selectively alterable channels (108, 110) and having imaging computations performed on a computing device 112 according to principles described herein. As described above, the imaging device 100 may be used to generate an image of internal tissue, bones, blood flow, or organs of human or animal bodies. Accordingly, the imaging device 100 may transmit a signal into the body and receive a reflected signal from the body part being imaged. Such imaging devices may include either pMUT or cMUT, which may be referred to as transducers or imagers, which may be based on photo-acoustic or ultrasonic effects. The imaging device 100 can be used to image other objects as well. For example, the imaging device can be used in medical imaging; flow measurements in pipes, speaker, and microphone arrays; lithotripsy; localized tissue heating for therapeutic; and highly intensive focused ultrasound (HIFU) surgery.

In addition to use with human patients, the imaging device 100 may be used to acquire an image of internal organs of an animal as well. Moreover, in addition to imaging internal organs, the imaging device 100 may also be used to determine direction and velocity of blood flow in arteries and veins as in Doppler mode imaging and may also be used to measure tissue stiffness.

The imaging device 100 may be used to perform different types of imaging in respective imaging modes. For example, the imaging device 100 may be used to perform one-dimensional imaging, also known as A-Scan, two-dimensional imaging, also known as B scan, three-dimensional imaging, also known as C scan, and Doppler imaging. The imaging device 100 may be switched to different imaging modes, including without limitation linear mode and sector mode, and electronically configured under program control.

To facilitate such imaging, the imaging device 100 includes one or more ultrasound transducers 102, each transducer 102 including an array of ultrasound transducer elements 104. Each ultrasound transducer element 104 may be embodied as any suitable transducer element, such as a pMUT or cMUT element or pixel. The transducer elements 104 operate to 1) generate the ultrasonic pressure waves that are to pass through the body or other mass and 2) receive reflected waves (received ultrasonic energy) off the object within the body, or other mass, to be imaged. In some examples, the imaging device 100 may be configured to simultaneously transmit and receive ultrasonic waveforms or ultrasonic pressure waves (pressure waves in short). For example, control circuitry 106 may be configured to control certain transducer elements 104 to send pressure waves toward the target object being imaged while other transducer elements 104, at the same time, receive the pressure waves/ultrasonic energy reflected from the target object, and generate electrical charges (receive signals) based on the same in response to the received waves/received ultrasonic energy/received energy.

In some examples, each transducer element 104 may be configured to transmit or receive signals at a certain frequency and bandwidth associated with a center frequency, as well as, optionally, at additional center frequencies and bandwidths. Such multi-frequency transducer elements 104 may be referred to as multi-modal elements 104 and can expand the bandwidth of the imaging device 100. The transducer element 104 may be able to emit or receive signals at any suitable center frequency, such as about 0.1 to about 100 megahertz. The transducer element 104 may be configured to emit or receive signals at one or more center frequencies in the range from about 3.5 to about 5 megahertz.

To generate the pressure waves, the imaging device 100 may include a number of transmit (Tx) channels 108 and a number of receive (Rx) channels 110. The transmit channels 108 may include a number of components that drive the transducer 102, i.e., the array of transducer elements 104, with a voltage pulse at a frequency that they are responsive to. This causes an ultrasonic waveform to be emitted from the transducer elements 104 towards an object to be imaged.

According to some embodiments, an ultrasonic waveform may include one or more ultrasonic pressure waves transmitted from one or more corresponding transducer elements of the imaging device substantially simultaneously.

The ultrasonic waveform travels towards the object to be imaged and a portion of the waveform is reflected back to the transducer 102, which converts it to an electrical energy through a piezoelectric effect. The receive channels 110 collect electrical energy thus obtained, and process it, and send it for example to the computing device 112, which develops or generates an image that can be displayed.

In some examples, while the number of transmit channels 108 and receive channels 110 in the imaging device 100 may remain constant, and the number of transducer elements 104 that they are coupled to may vary. A coupling of the transmit and receive channels to the transducer elements may be, in one embodiment, controlled by control circuitry 106. In some examples, for example as shown in FIG. 1, the control circuitry may include the transmit channels 108 and in the receive channels 110. For example, the transducer elements 104 of a transducer 102 may be formed into a two-dimensional spatial array with N columns and M rows. In a specific example, the two-dimensional array of transducer elements 104 may have 128 columns and 32 rows. In this example, the imaging device 100 may have up to 128 transmit channels 108 and up to 128 receive channels 110. In this example, each transmit channel 108 and receive channel 110 may be coupled to multiple or single pixels 104. For example, depending on the imaging mode (for example, whether a linear mode where a number of transducers transmit ultrasound waves in a same spatial direction, or a sector mode, where a number of transducers transmit ultrasound waves in different spatial directions), each column of transducer elements 104 may be coupled to a single transmit channel 108 and a single receive channel (110). In this example, the transmit channel 108 and receive channel 110 may receive composite signals, which composite signals combine signals received at each transducer element 104 within the respective column. In another example, i.e., during a different imaging mode, each transducer element 104 may be coupled to its dedicated transmit channel 108 and its dedicated receive channel 110. In some embodiments, a transducer element 104 may be coupled to both a transmit channel 108 and a receive channel 110. For example, a transducer element 104 may be adapted to create and transmit an ultrasound pulse and then detect the echo of that pulse in the form of converting the reflected ultrasonic energy into electrical energy.

The control circuitry 106 may be embodied as any circuit or circuits configured to perform the functions described herein. For example, the control circuitry 106 may be embodied as or otherwise include an application specific integrated circuit (ASIC), a field programmable gate array (FPGA), a system-on-a-chip, a processor and memory, a voltage source, a current source, one or more amplifiers, one or more digital-to-analog converters, one or more analog-to-digital converters, etc.

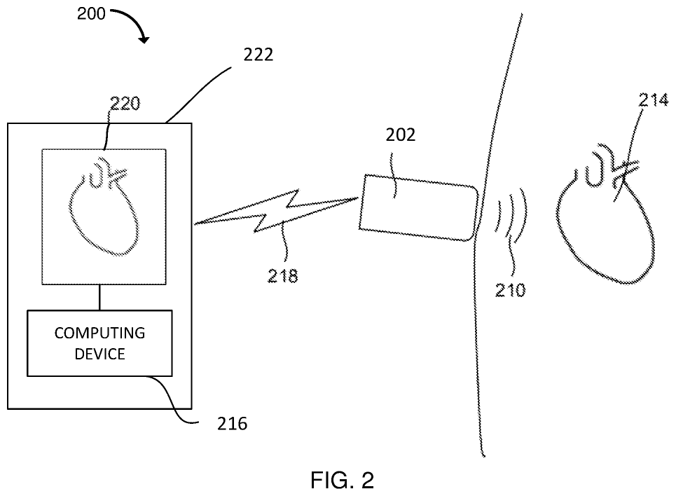

The illustrative computing device 112 may be embodied as any suitable computing device including any suitable components, such as a processor and a memory. A communication circuitry, battery, display, etc. (not shown in FIG. 1) may further be coupled to the computing device. In one embodiment, the computing device 112 may be integrated with the control circuitry 106, transducers 102, etc., into a single package or single chip, or a single system on a chip (SoC), as suggested for example in the embodiment of FIG. 1. In other embodiments, some or all of the computing devices may be in a separate package from the control circuitry, and the transducers, etc., as suggested for example in the embodiment of in FIG. 2 as will be described in further detail below.

Each transducer element may have any suitable shape such as, square, rectangle, ellipse, or circle. The transducer elements may be arranged in a two dimensional array arranged in orthogonal directions, such as in N columns and M rows as noted herein, or may be arranged in an asymmetric (or staggered) rectilinear array.

Transducer elements 104 may have associated transmit driver circuits of associated transmit channels, and low noise amplifiers of associated receive channels. Thus, a transmit channel may include transmit drivers, and a receive channel may include one or more low noise amplifiers. For example, although not explicitly shown, the transmit and receive channels may each include multiplexing and address control circuitry to enable specific transducer elements and sets of transducer elements to be activated, deactivated or put in low power mode. It is understood that transducers may be arranged in patterns other than orthogonal rows and columns, such as in a circular fashion, or in other patterns based on the ranges of ultrasonic waveforms to be generated therefrom.

FIG. 2 is a diagram of an imaging environment including an imaging system with selectively configurable characteristics, according to an embodiment. The imaging system of FIG. 2 may include an imaging device 202 and a computing system 222 which includes a computing device 216 and a display 220 coupled to the computing device, as will be described in further detail below.

As depicted in FIG. 2, the computing device 216 may, according to one embodiment, and unlike the embodiment of FIG. 1, be physically separate from the imaging device 220. For example, the computing device 216 and display device 220 may be disposed within a separate device (in this context, the shown computing system 222, physically separate from imaging device 202 during operation) as compared with the components of the imaging device 202. The computing system 222 may include a mobile device, such as cell phone or tablet, or a stationary computing device, which can display images to a user. In another example, as shown in FIG. 1 for example, the display device, the computing device, and associated display, may be part of the imaging device 202 (now shown). That is, the imaging device 100, computing device 216, and display device 220 may be disposed within a single housing.

A “computing device” as referred to herein may, in some embodiments, be configured to generate signals to at least one of cause an image of the object to be displayed on a display, or causing other processing of the received signals from the imaging device.

As depicted, the imaging system includes the imaging device 202 that is configured to generate and transmit, via the transmit channels (FIG. 1, 108), pressure waves 210 toward an object, such as a heart 214, in a transmit mode/process. The internal organ, or other object to be imaged, may reflect a portion of the pressure waves 210 toward the imaging device 202 which may receive, via a transducer (such as transducer 102 of FIG. 1), receive channels (FIG. 1, 110), control circuitry (FIG. 1, 106), the reflected pressure waves. The transducer may generate an electrical signal based on the received ultrasonic energy in a receive mode/process. A transmit mode or receive mode may be applicable in the context of imaging devices that may be configured to either transmit or receive, but at different times. However, as noted previously, some imaging devices according to embodiments may be adapted to be in both a transmit mode and a receive mode simultaneously. The system also includes a computing device 216 that is to communicate with the imaging device 100 through a communication channel, such as a wireless communication channel 218 as shown, although embodiments also encompass within their scope wired communication between a computing system and imaging device. The imaging device 100 may communicate signals to the computing device 216 which may have one or more processors to process the received signals to complete formation of an image of the object. A display device 220 of the computing system 222 may then display images of the object using the signals from the computing device.

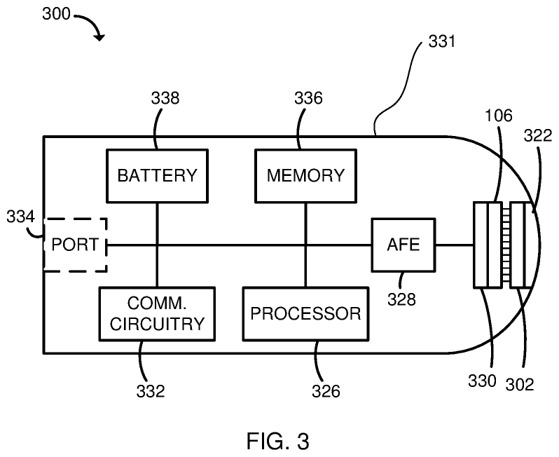

An imaging device according to some embodiments may include a portable device, and/or a handheld device that is adapted to communicate signals through a communication channel, either wirelessly (using a wireless communication protocol, such as an IEEE 802.11 or Wi-Fi protocol, a Bluetooth protocol, including Bluetooth Low Energy, a mmWave communication protocol, or any other wireless communication protocol as would be within the knowledge of a skilled person) or via a wired connection such as a cable (such as USB2, USB 3, USB 3.1, and USB-C, Ethernet) or such as interconnects on a microelectronic device, with the computing device. In the case of a tethered or wired, connection, the imaging device may include a port as will be described in further detail in the context of FIG. 3 for receiving a cable connection of a cable that is to communicate with the computing device. In the case of a wireless connection, the imaging device 100 may include a wireless transceiver to communicate with the computing device 216.

It should be appreciated that, in various embodiments, different aspects of the disclosure may be performed in different components. For example, in one embodiment, the imaging device may include circuitry (such as the channels) to cause ultrasound waveforms to be sent and received through its transducers, while the computing device may be adapted to control such circuitry to the generate ultrasound waveforms at the transducer elements of the imaging device using voltage signals. In such an embodiment, the computing device may process signals from the imaging device to construct images of the target using frames as discussed in more detail below, may select and configure transmit and receive channels, or may control the control circuitry to operate in one of a number of imaging modes, etc.

In another embodiment, the imaging device may include control circuitry to control a generation of the ultrasound waveforms at the transducer elements using voltage signals in order to cause the ultrasound waveform to be sent and received from the transducer elements, and may also generate electrical signals from the received ultrasound energy. In such an embodiment, the control circuitry of the imaging device may send the electrical signals generated from the received ultrasound energy to the computing device, which may process them in order to determine the target image to be generated. More generally, it should be appreciated that any suitable function disclosed herein may be performed by one or more circuitries, and that these circuitries may be housed in one physical device, or housed physically separately from each other, but communicatively coupled to one another.

FIG. 3 shows a schematic view of an imaging device and of internal components within the housing of the imaging device according to some embodiments, as will be described in further detail below.

As seen in FIG. 3, the imaging device 300 may include a handheld casing 331 where transducers 302 and associated electronics are housed. The imaging device may also contain a battery 338 to power the electronics. FIG. 3 thus shows an embodiment of a portable imaging device capable of 2D and 3D imaging using pMUTs in a 2D array, optionally built on a silicon wafer. Such an array coupled to an application specific integrated circuit (ASIC) 106 with electronic configuration of certain parameters, enables a higher quality of image processing at a low cost than has been previously possible. Further by controlling certain parameters, for example the number of channels used, power consumption can be altered and temperature can be changed. The imaging device 300 may be similar to imaging device 100 of FIG. 1, or to imaging device 202 of FIG. 2, by way of example only. As described above, the imaging device may include an ultrasonic medical probe. FIG. 3 depicts transducer(s) 302 of the imaging device 300. As described above, the transducer(s) 302 may include arrays of transducer elements (FIG. 1, 104) that are adapted to transmit and receive pressure waves (FIG. 2, 210). In some examples, the imaging device 300 may include a coating layer 322 that serves as an impedance matching interface between the transducers 302 and the human body, or other mass or tissue through which the pressure waves (FIG. 2, 210) are transmitted. In some cases, the coating layer 322 may serve as a lens when designed with the curvature consistent with focal length desired.

The imaging device 300 may be embodied in any suitable form factor. In some embodiments, part of the imaging device 300 that includes the transducers 302 may extend outward from the rest of the imaging device 100. The imaging device 300 may be embodied as any suitable ultrasonic medical probe, such as a convex array probe, a micro-convex array probe, a linear array probe, an endovaginal probe, endorectal probe, a surgical probe, an intraoperative probe, etc.

In some embodiments, the user may apply gel on the skin of a living body before a direct contact with the coating layer 322 so that the impedance matching at the interface between the coating layer 322 and the human body may be improved. Impedance matching reduces the loss of the pressure waves (FIG. 2, 210) at the interface and the loss of the reflected wave travelling toward the imaging device 300 at the interface.

In some examples, the coating layer 322 may be a flat layer to maximize transmission of acoustic signals from the transducer(s) 102 to the body and vice versa. The thickness of the coating layer 322 may be a quarter wavelength of the pressure wave (FIG. 2, 210) to be generated at the transducer(s) 102.

The imaging device 300 also includes a control circuitry 106, such as one or more processors, optionally in the form of an application-specific integrated circuit (ASIC chip or ASIC), for controlling the transducers 102. The control circuitry 106 may be coupled to the transducers 102, such as by way of bumps. As described above, the transmit channels 108 and receive channels 110 may be selectively alterable or adjustable, meaning that the quantity of transmit channels 108 and receive channels 110 that are active at a given time may be altered, for example based on the imaging mode of the imaging device. For example, the control circuitry 106 may be adapted to selectively adjust the transmit channels 108 and receive channel 110 based on whether the imaging device is to perform one-dimensional imaging, also known as A-Scan, two-dimensional imaging, also known as B scan, three-dimensional imaging, also known as C scan, Doppler imaging, sector mode imaging or linear mode imaging, and may be electronically configured under program control, for example by the computing device.

The imaging device may also include one or more processors 326 for controlling the components of the imaging device 100. One or more processors 326 may be configured to, in addition to control circuitry 106, at least one of control an activation of transducer elements, process electrical signals based on reflected ultrasonic waveforms from the transducer elements or generate signals to cause a restoration of an image of an object being imaged by one or more processors of a computing device, such as computing device 112 of FIG. 1 or 216 of FIG. 2. One or more processors 326 may further be adapted to perform other processing functions associated with the imaging device. The one or more processors 326 may be embodied as any type of processors 326. For example, the one or more processors 326 may be embodied as a single or multi-core processor(s), a single or multi-socket processor, a digital signal processor, a graphics processor, a neural network compute engine, an image processor, a microcontroller, a field programmable gate array (FPGA), or other processor or processing/controlling circuit. The imaging device 100 may also include circuit(s) 328, such as Analog Front End (AFE), for processing/conditioning signals, and an acoustic absorber layer 330 for absorbing waves that are generated by the transducers 102 and propagated towards the circuits 328. That is, the transducer(s) 102 may be mounted on a substrate and may be attached to an acoustic absorber layer 330. This layer absorbs any ultrasonic signals that are emitted in the reverse direction (i.e., in a direction away from coating layer 322 in a direction toward port 334), which may otherwise be reflected and interfere with the quality of the image. While FIG. 3 depicts the acoustic absorber layer 330, this component may be omitted in cases where other components prevent a material transmission of ultrasound in the reverse direction.

The analog front end 328 may be embodied as any circuit or circuits configured to interface with the control circuitry 106 and other components of the imaging device, such as the processor 326. For example, the analog front end 328 may include, e.g., one or more digital-to-analog converters, one or more analog-to-digital converters, one or more amplifiers, etc.

The imaging device may include a communication unit 332 for communicating data, including control signals or receive signals (from an imaging operation), with an external device, such as the computing device (FIG. 2, 216), through for example a port 334 or a wireless transceiver. The imaging device 100 may include memory 336 for storing data. The memory 336 may be embodied as any type of volatile or non-volatile memory or data storage capable of performing the functions described herein. In operation, the memory 336 may store various data and software used during operation of the imaging device 100 such as operating systems, applications, programs, libraries, and drivers.

In some examples, the imaging device 100 may include a battery 338 for providing electrical power to the components of the imaging device 100. The battery 338 may also include battery charging circuits which may be wireless or wired charging circuits (not shown). The imaging device may include a gauge that indicates a battery charge consumed and is used to configure the imaging device to optimize power management for improved battery life. Additionally or alternatively, in some embodiments, the imaging device may be powered by an external power source, such as by plugging the imaging device into a wall outlet.

Some embodiments provide a casing for a portable ultrasonic imaging device that is configured to protect the imaging device against at least one of some environmental stresses and mechanical shock, and that further optionally provides a backup or auxiliary source of power supply to enable the provision of power to the imaging device. The auxiliary source of power may include, for example, a rechargeable integrated battery for the casing, which may be charged in turn through a wired electrical connection using a vehicle or wall socket, using wireless power, or by way of energy supplied through solar panels.

As noted previously, for example in the context of FIG. 2 above, in some embodiments, the portable imaging device itself may not have an integrated display, either to display ultrasound images or to display a user interface to operate various operating modes of the portable imaging device. In some embodiments, such as the embodiment of FIG. 2, the portable imaging device may be communicatively coupled, through a wired and/or wireless connection, to a display device, such as a tablet or smartphone, to provide this image display and user interface functionality. There may be circumstances, particularly in the field or in remote deployments of the portable imaging device, when the operator of the device may not have a smartphone, tablet or other display device to view ultrasound image output from the imaging device, or to input operating controls through a visually-based, touch screen user interface. The utility of the MUT-based portable imaging device is not to be limited, especially in emergency situations, by a dependency on the availability of a tablet or a smartphone or other display device to the operator during operation of the imaging device, much less to such dependability where the display device ought to be configured to be communicatively coupled with the ultrasound portable imaging device. In addition, in an unexpected emergency situation requiring the MUT-based ultrasound portable imaging device, the operator's tablet or smartphone device may not be sufficiently charged, even if available and configured for communicative coupling to the imaging device, to allow display as would be required by an imaging round of the imaging device.

Furthermore, there may be circumstances when ultrasound image data either needs to be stored remotely for later analysis or perhaps, due to emergency circumstances, may require further examination or analysis by a physician or ultrasound imaging expert not physically present at the location where the imaging device is being used. It would be desirable for a first responder operating an ultrasound imaging portable imaging device at the scene of an accident to be able to transmit the imaging data to a physician at a location remote from the accident location, such that the physician can review the image data in real- or near-real time. The physician in this example would be enabled to analyze the data for at least a preliminary analysis, and advise the first responders regarding injuries revealed by the ultrasound image. The physician might be able to render time-sensitive treatment information to the first responders for the patient, well before the patient arrives at the hospital. Alternatively, the physician (or other ultrasound imaging expert) at the hospital may advise the first responders to image other parts of the patient's body, instruct or advise on alternative imaging modes or methods, in order to gather additional useful ultrasound imaging data. Thus, a valuable feature to deploy with the MUT-based ultrasound portable imaging device according to one embodiment is to provide a two-way wireless communications capability to transmit imaging data and receive remote feedback, advice or instruction.

According to another embodiment, the wireless communications capability may allow an upload of ultrasound image data, along with other information or metadata associated with the image (including, without limitation, patient identifying information (e.g., name, age, gender), date of image, geographic location of image capture, etc.) to a remote storage or server for future retrieval and analysis.

An additional useful feature of some embodiments, for example as an alternative or in addition to the image transmission capability, is the provision, for the imaging device, of a local data storage capability, such as a non-volatile memory. Such data storage capability may be useful particularly in instances when a wireless network is not immediately available for transmitting images to a remote end-user or data storage facility. The local non-volatile memory may store ultrasound image data for immediate retrieval and analysis, or for later upload to an alternative data storage system, such as cloud storage.

According to some embodiments, one or more of the above advantageous embodiments, including, without limitation, a backup battery recharging capability, wired or wireless communication capability, display capability, and local data storage capability, may be packaged in a form factor that minimizes size and weight, and that is integrated inside an enclosure providing physical protection against mechanical and thermal shock to its enclosed components, including the MUT-based ultrasound portable imaging device itself.

According to one embodiment, a protective case or casing for a portable MUT-based portable imaging device is provided. The protective casing may have a rigid exterior, a cushioned or padded interior to protect the contents of the case, including in particular the ultrasound portable imaging device stored or to be stored within.

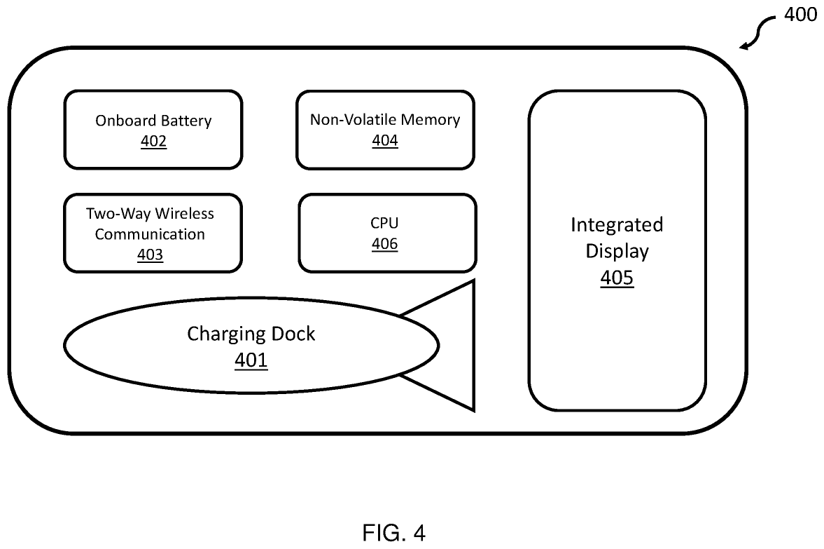

As shown in the functional block diagram of FIG. 4, an embodiment of a protective casing 400 may further include an integrated charging dock 401 with an onboard battery 402, a wireless communication circuitry 403, non-volatile memory 404, and/or an integrated touch screen display 405, or any combination thereof. A CPU or processor 406 may also be integrated into the protective casing in order to coordinate the functionality and interoperability of onboard battery 402, two-way wireless communication subsystem 403, non-volatile memory 404, and/or integrated touch screen display 405. Some or all of the signal processing functionality of the computing device 112 of FIG. 1 or 216 of FIG. 2 may further be offloaded onto processor 406 during or after operation of the imaging device to obtain images of a target. For example, the processor 406 may perform all of a parts of the computing device 216 of FIG. 2.

Various features of a casing according to some embodiments will be addressed serially below.

Impact-Resistant Exterior

According to some embodiments, a protective casing may include an impact-resistant material, such as an impact-resistant plastic. The impact resistant material may include a rigid material, or a flexible material, such as rubber or a rubber-like material. The protective casing may be configured to provide physical protection for the MUT-based portable imaging device and its accessories against mechanical stresses, mechanical shock/impact damage as might be experienced if the casing containing the MUT-based portable imaging device were dropped, or a heavy object were to be dropped onto the protective casing containing the MUT-based portable imaging device.

Security Against Unauthorized Access

According to some embodiments, the protective casing may provide physical security against unauthorized access and use of the MUT-based portable imaging device. For example, the protective casing may include a lock. A lock as used herein refers to a mechanical or electronic fastening device that is released by a physical object (such as a key, keycard, fingerprint, RFID card, security token, etc.), by supplying secret information (such as a number or letter permutation or password), or by a combination thereof.

Organization for Accessories

According to some embodiments, the protective casing may also include one or more containers or compartments for accessories of the imaging devices, and may thus also provides added organization and portability for such accessories, including associated cords, cables, gel packets, and/or extra batteries. With the portable imaging device and its associated accessories stored within, the protective casing enables easy transportation and shipment to remote locations.

General Design

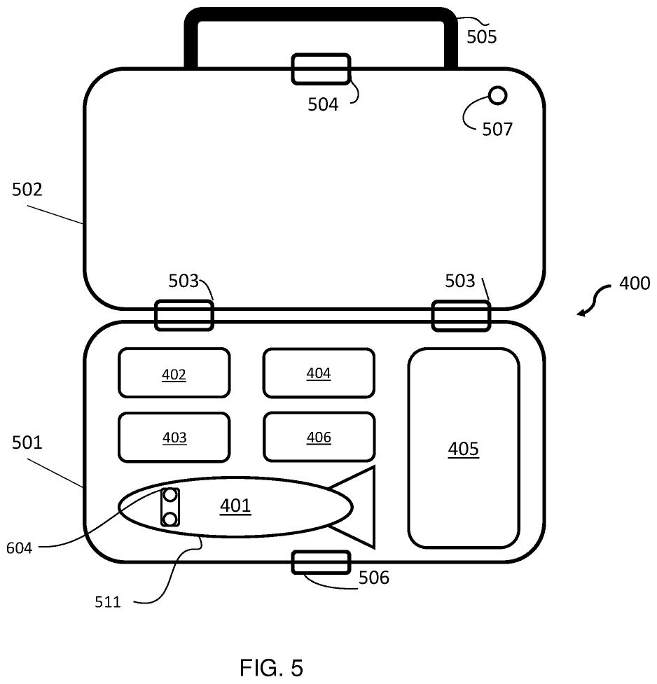

Referring now to FIG. 5, according to some embodiments, the protective casing 400 may include a clamshell or briefcase-style design including two portions, a base 501 and a lid 502. In the shown embodiment, the base portion and the lid are connected by one or more hinges 503 that allow the casing to pivot open and closed, although embodiments include within their scope a base and lid portion that are: (1) connected to one another at corresponding side edges thereof so that they can pivot with respect to each other in order for the casing to close, such as by way of hinges, a pliable material that may be folded, or any other suitable mechanism; and/or (2) configured to be separated from one another when the casing is open, and to be connected to one another to close the casing through any suitable mechanism, such as latches and or snap closures.

The inner material of the casing may be made of a compliant material, such as a compliant plastic, foam or rubber material that may provide a conformal, shock absorbing recess or cutout for the imaging device and possibly for its accessories, plastic or padding with a cutout or cradle to retain and protect an ultrasonic portable imaging device therein.

Casing 400, for example at base 501, may also house charging contacts 604 within integrated charging dock 401, the contacts leading to/from associated circuitry (not shown) to supply power to an imaging device in the charging dock 401. The charging dock 401 may be configured to be supplied with power from any power source, such as an AC source of power through socket, or by way of the onboard battery 402. The charging or electrical contacts are to couple the portable imaging device to a backup or auxiliary power source (such as battery 402) to charge the portable imaging device, such as integrated battery 338 of imaging device 300, in the event that the integrated battery, such as battery 338 of the imaging device 300 of FIG. 3, should be at a low power level or no power.

Casing 400, for example at base 501, may also include wireless communication circuitry 403, and/or ports or wires for wired coupling to another device, such as to the imaging device. The wireless communication circuitry 403 may include circuitry to cause communication wirelessly, such as by using a wireless communication protocol, e.g. an IEEE 802.11 or Wi-Fi protocol, a Bluetooth protocol, including Bluetooth Low Energy, a mmWave communication protocol, a cellular communication protocol such as a Third Generation Partnership Project (3GPP) 2G, 4G, 5G or 6G communication protocol, an Internet-of-Things protocol such as Thread or Zigbee, or any other wireless communication protocol as would be within the knowledge of a skilled person). The port(s) (into which one or more wires may be plugged) or the wires if provided, may for example use communication protocols such as Ethernet, RS-232, RS-485, UART, USART, USB2, USB 3, USB 3.1, and/or USB-C.

Casing 400, for example at base 501, may also include non-volatile memory; or an integrated display; or any combination of the foregoing. The lid 502 may also include internal foam, plastic or padding to further protect an ultrasonic portable imaging device. The lid 502 may also house some of the features, such as wireless communication circuitry 403, non-volatile memory 404, or an integrated display, such as an integrated touch-screen display, in order to distribute components that enable various functions and features within the protective casing in a space-efficient manner. Alternatively, some components for specific functionality may be distributed between and housed within the base 501 and the lid 502. For example, with respect to wireless communication functionality (described in greater detail herein), some components such as an application processor and/or baseband processor of wireless communication circuitry 403 may be housed in the base 501, whereas other wireless communication-related components of circuitry 403, such as one or more antennae structure required for wireless communication, may be housed within the structure of the lid 502.

The protective casing may further include one or more latches 504 that, when engaged, maintain the protective casing in a closed position. The protective casing may optionally include one or more carrying handles 505 or adjustable straps. The handles or straps provide a way for the portable imaging device owner or user to transport the protective casing with relative convenience and ease.

The protective casing may optionally include one or more locks 506 that limit access to the contents of the casing only to those with authorized access. One of ordinary skill in the art would appreciate that a different types of locks for effective physical security of the protective casing contents are possible, including key locks, combination locks, fingerprint-reader based locks, or other locks that utilize biometric methods, as a basis for authorized access.

The exterior or shell of the protective casing is comprised of a durable, impact-resistant material. For example, the protective casing exterior may be comprised of a molded plastic that exhibits mechanical strength, stiffness and abrasion resistance properties to protect its contents from having been dropped from a height, or from a relatively high impact force delivered in a short period of time.

The protective casing may optionally include a waterproof gasket (not shown in FIG. 5) that creates a waterproof seal when the protective casing is fully closed. For example, a waterproof gasket seated along the periphery of one of the pivoting portions of the casing (e.g., base 501) of the casing where it makes contact with the periphery of the other pivoting portion of the casing (e.g., lid 502) creates a waterproof barrier when the casing portions are pressed together, preferably when one or more latches 504 or locks 506 are engaged to create additional sealing pressure.

The protective casing may optionally include a pressure release or pressure equalization valve 507 to allow the internal pressure of the closed and sealed casing to equalize with external pressure, to relieve the casing of stress-inducing pressures.

Protective Interior Cushioning

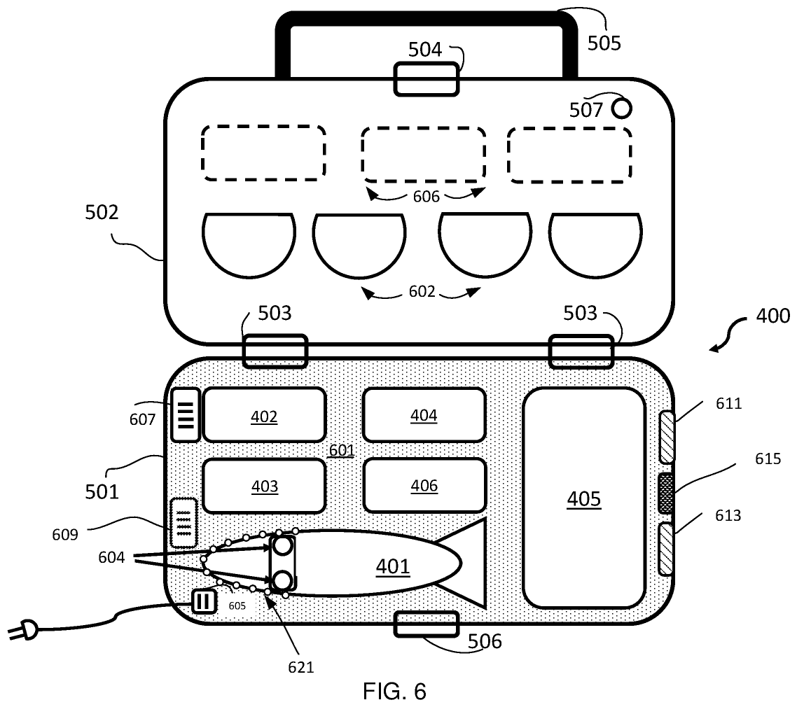

Referring now to FIG. 6, the protective casing 400 includes a protective interior material 601 to provide stability and cushioning of casing contents against mechanical forces (e.g., a drop against a hard surface) or a buildup of external pressure on the case. The protective interior material 601 may include a compliant material, such as at least one of open-cell foam, closed-cell foam, hybrid open-cell and closed-cell foam, one or more gel-filled cushions, molded plastic, or any other compliant material, such as a rubber material that may define a recess therein substantially conformal to an outer shape of the imaging device, the recess providing shock absorption for the imaging device. More similar recesses may be provided for accessories of the imaging device, and may provide similar shock absorbing benefits for its accessories. In the case of a foam material for the protector interior material, a precision cutout of the foam material conforming at least approximately to the geometry of the portable imaging device may be provided as the recess to enable the portable imaging device to be stored in a relatively fixed position with a minimum of unintended movement or shifting during transportation. Alternatively, the interior of the protective casing may include form-fitting plastic molded to define one or more recesses to the approximate geometry of the portable imaging device and possibly its accessories, to secure the portable imaging device and its accessories in a relatively fixed position during transportation. For example, the interior plastic may be molded such that, with a degree of pressure, the portable imaging device snaps into it and is retained by a molded cavity that approximates the geometry of the portable imaging device. The interior of the protective casing may also include clips, straps, bands, or magnets, that either alone or in conjunction with the interior foam or molded plastic, secure the internal contents in place.

The protective casing interior may optionally include compartments, such as integrated compartments, for accessories, such as pockets or pouches 602, to hold miscellaneous items for the imaging device, such as data transfer cords, charging cables, gel packets, alcohol pads, extra batteries, and so on. The integrated pockets may be open or include some method of closure, including for example, zippers, snaps, drawstrings, hoop and loop tape, or elastic drawstrings.

The protective casing interior may further include a disinfecting mechanism at a location where the imaging device is to be stored, such as at the recess adapted to receive the imaging device therein. According to one embodiment, the disinfecting mechanism may include one or more ultraviolet disinfecting light sources 621 located at the walls of the recess and adapted to direct disinfecting light toward portions of the imaging device to be disinfected. The disinfecting light sources 621 may be coupled to the processor 406, or to its own dedicated processor, and may for example be activated as soon as the imaging device is positioned in the recess of the casing. For example, a sensor, such as a touch sensitive mechanism, may be activated when the imaging device is positioned within the recess, and/or when the casing lid is closed, in order to activate disinfection of the same through a powering up of the disinfecting lights. The processor 406 or dedicated processor may be configured to keep the lights on for a predefined period of time.

Integrated Charging Dock

Referring still to FIG. 6, in a preferred embodiment, the protective casing may include an integrated charging dock 401 including charging contacts 604 for the ultrasonic portable imaging device. When the portable imaging device is seated within the casing, the portable imaging device may simultaneously be in electrical contact with one or more charging contacts 604. In an alternative embodiment, the protective casing may include circuitry and components for inductive or resonant charging of the imaging device when the imaging device is within the casing, such as using any one of wireless charging protocols, including at least one of AirFuel Alliance (AFA)'s Rezence, or Qi, or other wireless charging mechanisms. Whether using direct electrical contact or wireless charging, when the portable imaging device's rechargeable integrated battery remaining power balance falls beneath a predetermined threshold, the charging dock may be used to supply power to the portable imaging device according to a predetermined charging power profile to recharge the portable imaging device's rechargeable batteries. The recharging operation of the integrated charging dock ceases when the portable imaging device rechargeable battery reaches a predetermined threshold.

During the recharging operation in the protective casing, the integrated charging dock may transfer power to the portable imaging device rechargeable battery either from an external power source, such as from a household AC outlet or vehicle outlet, from a separate power storage unit (e.g., an onboard battery) 402 that itself is integrated into the protective casing and itself has been charged beforehand, through charging coils in the casing and in the imaging device, respectively, for inductive and/or resonant charging. When charging from an external power source, a charging power supply may be connected between an external electrical outlet (e.g., AC outlet) and a port 605 (e.g. USB port) located within the protective casing. In addition, the onboard battery 402 integrated into the protective casing, may be recharged by an external power source, such as from a household AC outlet or vehicle outlet, and is of sufficient capacity to hold enough charge to enable at least one recharge of the portable imaging device rechargeable battery.

According to some embodiments, the onboard battery may be recharged through solar panels 606 that either are integrated into the external surface of the protective casing, or are separately detachable and deployable for recharging the onboard battery, and then may be stored away separately inside the protective casing. The optional solar panels provide a source of recharging power for the onboard battery in instances when there is no local supply of electricity to recharge the onboard battery, such as may be the case in remote field deployment situations for the ultrasonic portable imaging device.

According to some embodiments, a remaining battery power or charge status indicator 607 associated with the onboard battery indicates how much power remains in the onboard battery. Optionally, another charge status indicator 609 may be provided on the casing to indicate the charge on the imaging device when the imaging device is encased within the casing. Any of the charge status indicators may include one or more light emitting diodes that indicate relative battery power level, and varying the color of the one or more light emitting diodes, or flashing the light emitting diodes in various flashing patterns (e.g., slow flashes and rapid flashes) may further indicate the charge status of the onboard battery 102. According to one embodiment, the charge status indicators 607 and 609 may further indicate to a user whether the onboard battery 402 or 338, respectively, are being charged (for example, from an external AC outlet), is fully charged, and/or whether the onboard battery is being discharged as part of a portable imaging device rechargeable battery charging process.

According to some embodiments, the protective casing may also include a jack or charging port 611, such as a USB-C or other charging port, to enable charging of the battery 402 and/or of other peripheral devices (not shown in FIG. 6) that may be used in conjunction with the ultrasonic portable imaging device.

According to some embodiments, the protective casing may further include a jack or charging port 613 to allow a peripheral device, such as a tablet, a smartphone, a peripheral display device, to be charged through the casing, using the same power sources as described above for the imaging device, such as by way of battery 402, or by way of the casing's connection to an AC electrical power source.

According to some embodiments the protective casing may further include a jack or data transfer port 615 to allow data transfer to or from a peripheral device, such as a tablet, a smartphone, or auxiliary memory device such as USB enabled memory card, or other external memory, to be sent or received from or to the memory 404 of the casing. In such a situation, it may be possible to transfer ultrasound imaging data from the non-volatile memory to an auxiliary memory, for example for transportation, for wireless or wired transfer to another memory location, in instances where the memory 404 of the casing may not have enough storage space for all captured ultrasound imaging data, etc. The transfer of ultrasound imaging data may occur from an auxiliary memory to the memory 404 of the casing for example in instances where the memory 404 may not have enough space for all captured ultrasound data, but where display 405 may be useful in displaying ultrasound images corresponding to the data for each set of captured ultrasound data (for example per patient, per organ, per location, etc.).

As noted previously, the portable ultrasonic portable imaging device that is stored within the protective casing may be coupled, either by cable or through a wireless signal connection (e.g., WiFi, Bluetooth, mmWave, etc.), to a tablet device or smartphone-style device to display ultrasound portable imaging device images and imaging data. Because tablet and smartphone devices themselves operate using their own rechargeable batteries, a charging port (e.g., USB) 611 to enable a charging connection between the tablet or smartphone device and the power source for the casing would be useful, to recharge the tablet or smartphone battery to enable more prolonged operation (e.g., image display) in conjunction with the ultrasonic portable imaging device. Any associated cables (e.g., USB to USB-C, or USB to Lightning) may be optionally stored within the integrated pockets, pouches or compartments 302 inside the protective casing.

Wireless Communication

The portable nature of MUT-based portable imaging devices provides opportunities for remote deployment and ultrasound imaging data collection. In certain circumstances, however, there are additional benefits to transmitting ultrasound image data, in real-time or near real-time, to a location remote from the site where the portable imaging device actually is deployed. There may be circumstances when ultrasound image data either needs to be stored remotely for later analysis or perhaps, due to emergency circumstances, requires further examination or analysis by a physician or ultrasound imaging expert not physically present at the location of where the ultrasound imaging is taking place.

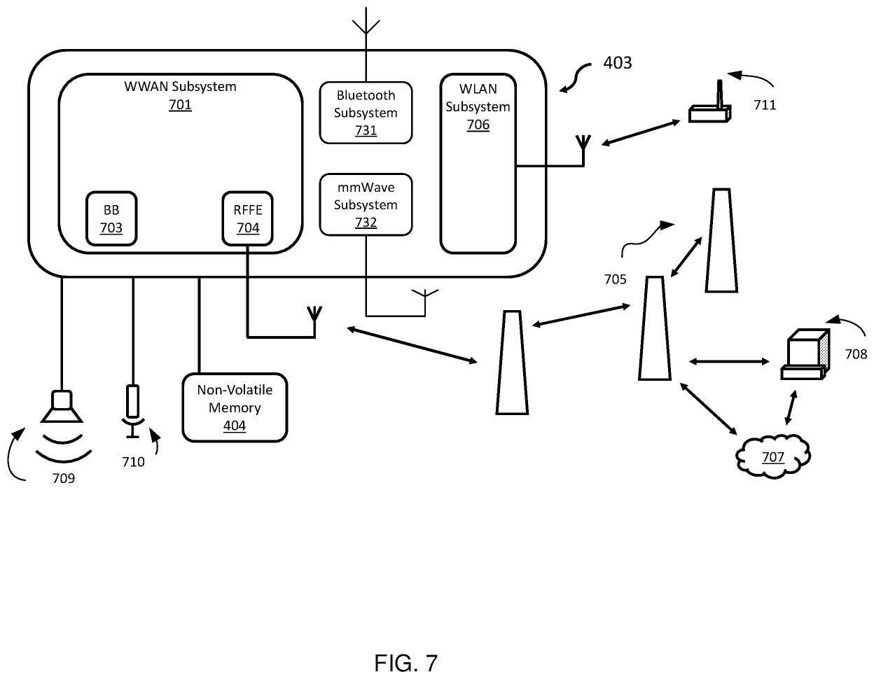

With reference to FIG. 7, according to some embodiments, a two-way (uplink and downlink) wireless communications capability to transmit imaging data and receive remote feedback, advice or instruction may be incorporated within the protective casing by way of one or more wireless transceivers 403. According to some embodiments, the protective casing 400 may include two-way wireless communication subsystem 403 as noted previously, which may include at least one of an integrated wireless wide area network (WWAN) communication circuitry 701, or integrated wireless local area network (WLAN) communication circuitry 706, an integrated Bluetooth and/or Bluetooth low energy communication circuitry 731, an integrated mmWave (such as an IEEE 802.11ay or 802.11ad) communication circuitry 732. In generation, one or more wireless transceivers, as noted above, may include one or more transceiver compliant with an IEEE 802.11 or Wi-Fi protocol, a Bluetooth protocol, including Bluetooth Low Energy, a mmWave communication protocol, a cellular communication protocol such as a Third Generation Partnership Project (3GPP) 2G, 4G, 5G or 6G communication protocol, an Internet-of-Things protocol such as Thread or Zigbee, or any other wireless communication protocol as would be within the knowledge of a skilled person.

According to some embodiments, each of the WWAN communication circuitry 701, the WLAN communication circuitry 706, the Bluetooth communication circuitry 731 and the mmWave communication circuitry 732 may include a baseband processor, a radio frequency (RF) circuitry and a RF front end module (FEM) to enable wireless communication in the uplink and downlink directions. The baseband circuitry 703 and RF FEM 704 are shown for the WWAN communication circuitry 701, although it is understood that, as noted above, compatible baseband processors, RF circuitries and FEMs may be provided for the WLAN, Bluetooth and/or mmWave circuitries as would be recognized by a skilled person. Each of the wireless communication circuitries may be connected, by way of their FEMs, to one or more associated antennas, and some of the communication circuitries may even share antennas (not shown).

According to some embodiments, a MUT-based portable imaging device may be configured to transmit its ultrasound imaging data via a wireless communication circuitry (e.g. communication 332 in FIG. 3) to wireless communication circuitry 403 within the protective casing, whereupon the imaging data received at the protective casing 400 may be at least one of: (1) processed by the processor 406 and caused to be sent to a display, either a display external to the casing, or to a display that is integrated with the casing, such as display 405; or (2) re-transmitted through a wireless or wired connection to a desired destination, such as an auxiliary memory or to a remote cloud storage 707 (for example in some of the scenarios described in relation to data port 615), and/or to a remote computer workstation 708 including or connected to a display, where it may be viewed by a remotely-located physician or ultrasound imaging analyst. The operation of the wireless communication circuitry 403 within the protective casing may be powered by the onboard battery 102. In this manner, the wireless communication capability provided by the protective casing may, in real- or near real-time, stream imaging data to an auxiliary or remote destination.

Because the wireless communication circuitry 403 of the protective casing may be capable of transmitting in both the uplink and downlink directions, in addition to transmitting imaging and other data in the uplink direction, the protective casing may be capable of receiving data in the downlink direction. For example, in response to ultrasound imaging data transmitted through the uplink, an individual receiving the imaging data at a transmission remote endpoint (e.g., a remote computer workstation 708 at a hospital) may want to provide verbal directions or guidance to the operator of the ultrasound portable imaging device. Such voice or text data may be received at the protective casing 400 by the integrated wireless communication circuitry 703 and output through optional speaker 709 that is onboard and integrated into the protective casing 400, or output through the display 405 in the form of text. The individual receiving the imaging data at the remote transmission endpoint 708 may want to send responsive textual or graphical information to the operator of the ultrasound portable imaging device, such as written instructions, image annotations, and so on. Textual and/or graphical information also may be received at the protective casing by the integrated wireless communication circuitry 403 and output though the optional onboard and integrated display 405, as already described herein. An optional microphone 710 that is onboard and integrated into the protective casing 400, or that may be connected to the would further enable two-way voice communication with an individual located at a remote transmission endpoint 408, such that the portable imaging device operator can communicate by voice with, for example, a remotely-located physician or ultrasound imaging analyst.

The WLAN communication circuitry 406 within the protective casing 400 may provide for WLAN communications according to one or more WLAN standards currently known, such as WiFi (IEEE 802.11) or according to one or more WLAN standards enabled by future WLAN standard-setting efforts. For example, a built-in WiFi functionality would enable wireless communications between the protective casing 400 of the present invention and a WLAN-enabled router 711 (e.g., WiFi router), for wireless communication of data with remote locations in the uplink and downlink directions. A built-in Bluetooth functionality would enable wireless communications between the protective casing of the present invention and other wireless communications-enabled medical devices (not shown in FIG. 7), such as a pulse oximeter or blood pressure monitor.

The ability to transfer data from the casing to another device, such as a remove computing system including a remote display, allows advantageous use of the imaging device in critical or life threatening scenarios in locations where a medical professional may not be available, and where the imaging data may be time sensitive and ripe for analysis and recommendations in the form of real-time diagnoses, feedback, and treatment guidance by the medical professional.

Non-Volatile Memory

Referring still to FIG. 7, according to some embodiments, the protective casing 400 includes onboard non-volatile memory 404. The amount of onboard non-volatile memory may be dependent on the final configuration of the protective casing and the anticipated extent of use of the imaging device without a need to recharge the onboard battery 402. In some embodiments, the amount of storage of the non-volatile memory may be based on the total battery capacity of the on board battery 402 and of the imaging device's battery 338. In some embodiments, the amount of storage of the non-volatile memory may be based on the availability of wireless communication circuitry of the casing. For example, the protective casing may include onboard non-volatile memory sufficient to store ultrasound imaging data for eventual offload and transfer to another data storage location (such as cloud storage 707). As another example, if the protective casing 400 either lacks wireless communication circuitry 701, or if the wireless communication circuitry 403 is unable to access an available wireless communications network, the onboard non-volatile memory 404 integrated into the protective casing may retain ultrasound image data until the protective casing with its stored imaging data is physically delivered to another location for data offload (e.g., a hospital), or until the wireless communication circuitry is able to access an available wireless communications network.

Onboard non-volatile memory 404 also may be utilized to store software instructions, such as that for operating the ultrasonic portable imaging device in various alternative operating modes, or for rendering a user interface on an optional onboard and integrated display, as further described herein. The onboard non-volatile memory 404 may also be used to store signal processing applications and algorithms to allow processor 406 to process and display the ultrasound imaging data in a manner selected by the user. Other software that may be stored in the onboard non-volatile memory 404 may include voice recognition software, to enable various features integrated into the protective casing 400 to be operable by user voice command. Onboard non-volatile memory 104 also offers the possibility of future software updates that may be released by ultrasound portable imaging device manufacturers or third party vendors, including updates that may “pushed” by the manufacturer, or be downloaded by a user, through a wireless communications network or through data port 615.

Processor

Integrated processor 406 may be adapted to perform one or more functionalities of a computing device, such as computing device 216 of FIG. 2, or of the control circuitry, such as control circuitry 106 of FIG. 1. For example, the processor 406 may be configured to generate signals to at least one of cause an image of the target being imaged to be displayed on a display, such as display 405 or a display coupled to the casing either through a wired or a wireless connection, or causing other processing of the received signals from the imaging device. The computing device may further, optionally control an activation of transmit and/or receive channels of the imaging device, for example by way of a wired or wireless connection.

According to some embodiments, the processor 406 may control the imaging device to operate in various imaging modes, such as a one-dimensional imaging mode, also known as A-Scan, a two-dimensional imaging mode, also known as B scan, a three-dimensional imaging mode, also known as C scan, and/or a Doppler imaging mode. The processor may further control the imaging device to operate in a linear mode or a sector mode, as described above. The processor 406 may therefore cause the imaging device 400 to be switched between two or more imaging modes.

According to some embodiments, the processor 406 may determine to change the imaging mode of the imaging device based on a determination that the imaging device or any portion thereof have exceeded one or more predetermined operating temperature thresholds

According to some embodiments, the processor 406 may be adapted to implement a feature identification algorithm to identify a target being imaged by the imaging device based on the signals from the imaging device, for example using image recognition software, including for example machine learning, and communicate the identification to a user.

According to some embodiments, the processor 406 may generate signals to cause image-related communication to a user regarding image data from the imaging device, the communication including, by way of example, at least one of markings/annotations/colors on the image, voice or text communication regarding the image to allow the user to interpret the image and to determine next steps regarding the target being imaged. For example, such next steps could include a determination as to a procedure to be performed on a patient undergoing imaging, as to next imaging steps, etc.

Integrated Touch Screen Display

Many portable MUT-based ultrasound portable imaging devices do not include an integrated display to show ultrasound images to a user, but rather rely on a tethered tablet device or smartphone device as a means for displaying images. In such a configuration, the tablet device or smartphone device may be connected to the portable MUT-based portable imaging device either with a wire cable or wireless communications connection for the exchange of imaging data with the tablet or smartphone device.

However, in instances where an auxiliary display, such as through a tablet, smartphone or other computer, may not be feasible, for example where such a device is not available in the field, or where use of the portable imaging device either does not have a separate tablet or smartphone display device, or when such display device may lack sufficient battery charge to function adequately as an imaging display. Display 405 of casing 400 provides a useful backup image display capability.

Referring back to FIG. 4, according to some embodiments, onboard display 405 may be integrated into the protective casing 400. The onboard display 405 does not need to be large, and its overall dimensions should be sufficient to display ultrasound images in a visually useful form (i.e., if the screen is too small to render important or user-recognizable ultrasound image features, then its usefulness is more limited). On the other hand, the onboard display 405 should not be so large as to adversely impact the overall dimensions, weight, and other features of the protective casing 400. According to some embodiments, the onboard display 405 may be powered by the onboard battery 102. According to another embodiment, the onboard display 405 may also be powered when the protective casing 400 is plugged into an AC power supply or other outside power supply, like a vehicle power supply, such as through port 605 or charging port 611.

According to another embodiment, the onboard display 405 may include a touchscreen and therefore be touch sensitive to enable the user to select user-selectable features and functions of the portable MUT-based ultrasound as presented on the display 405, for example, through on-screen menus. The touch sensitive nature of the screen may also enable the user to input textual information relating to the ultrasound images, such as an identity of the imaged subject, date, imaging location and so on. According to some embodiments, the user may call up or access one or more ultrasound images of interest stored in the onboard non-volatile memory 404 to display on the onboard display 405, and the user may add associated text, such as notes, other markings, or other supplementary data, through a virtual keyboard on the touch sensitive onboard display 405. The added text may then be stored in the onboard non-volatile memory 404 such that the added text is associated with the one or more specific images of interest.

According to some embodiments, the onboard display 405 may be fixed in-place in the protective casing. According to an alternative embodiment, the onboard display 405 viewing position may be pivoted to alter the display angle for improved viewing. According to yet another embodiment, the onboard display 405 may be a peripheral device of the casing, and may be coupled to the circuitry of the casing through a data port, such as data port 615, in order to receive data from the non-volatile memory 404, and to display images relating thereto, and to further send display-related data back to the non-volatile memory for storage.

Electrocardiography Subsystem

According to some embodiments, an electrocardiography subsystem for producing an electrocardiogram (ECG), i.e., measuring the electrical activity of the heart, may be integrated into protective casing 400. This ECG subsystem may include a set of electrodes that may be stored within the protective casing 400. The ECG electrodes may transmit their respective signals (electrical changes) to the ECG subsystem within the protective casing for further signal processing. The ECG subsystem is comprised of circuitry to receive, process, store or display (or any combination of the foregoing) the electrode signals, which circuitry may be part of processor 406, or separate from the same. According to some embodiments, the measured ECG electrode signal, in unprocessed or processed format, may be stored in the onboard non-volatile memory 404 for future retrieval and analysis. Processed ECG signals may also be output to the onboard display 405, where an image corresponding to the same may be viewed by the operator. Processed ECG signals may also be transmitted via wireless communication circuitry 403 to remote storage (such as remote cloud storage 707) or to a remote computer workstation 708 (where it may be viewed by a remotely-located physician or ECG specialist).

Ultraviolet Sanitizing Subsystem

According to some embodiments, an ultraviolet (UV) sanitizing subsystem or disinfecting mechanism, such as one including disinfecting lights 621, may be integrated into protective casing 400. In particular, the protective casing 400 may include light emitting diodes 621 to emit (LED) UV radiation to disinfect, sterilize or sanitize the ultrasound imaging portable imaging device while the portable imaging device is resting in charging dock 401. The UV sanitizing subsystem UV LEDs may, as shown in FIG. 6, be integrated either into the surface of the charging dock 401, in a portion and location of lid 202 such that the UV energy emitted by the UV LEDs reach the surface of the ultrasound imaging portable imaging device secured in the charging dock 401 when the protective casing 400 is closed, or both. The UV sanitizing subsystem may be powered by onboard battery 602.

The intensity and duration of UV energy emitted by the UV LEDs may be controlled, as noted previously, through circuitry integrated in the protective casing 400, such as through processor 406, or through a dedicated disinfecting mechanism processor, or a combination of both. Optionally, the UV sanitizing subsystem may activate only when the protective casing 400 is fully closed (as sensed by circuitry determining through, for example, hinges 503, lock 504 or other electrical contacts between base 501 and lid 502 that protective casing 400 is closed). Optionally, the protective casing 400 manufacturer or provider may pre-program a minimum time length or duration of UV sanitization of the portable imaging device. Optionally, the user may activate or deactivate, or alter the operating parameters of the UV sanitizing subsystem (such as duration of UV sanitization) through, for example, onboard integrated display 405. Preferably, the UV LEDs emit UV energy in at least the UV-C portion of the spectrum, at or around 254 nm wavelength, to eliminate pathogens such as bacteria, viruses, mold and so on from the surfaces of the ultrasound imaging portable imaging device, thereby disinfecting and sanitizing the portable imaging device prior to its next use.

Point-of-Care Kiosk

According to some embodiments, the protective casing 400, including the subsystems disclosed previously herein, may be used as part of a point-of-care kiosk that may be deployed, for example, at a pharmacy or health clinic. The kiosk itself may have an integrated display separate and larger than the integrated display 405 of the protective casing for better viewing of ultrasound images for the operator and patient. In this particular embodiment, the protective casing may integrate or connect with the kiosk through a direct electrical contact mechanism, like a docking port, or through a cable, for example by way of jack 615. Ultrasound images collected at the point-of-care kiosk could be stored locally in the onboard non-volatile 404 and/or uploaded through the two-way wireless communication subsystem 403 to remote storage 707 and/or remote computer workstation 708 as described previously. In-between scanning sessions, or doing non-operating hours, the portable imaging device could be secured in the protective casing, be recharged, and be disinfected in the meantime by an ultraviolet sanitizing subsystem.

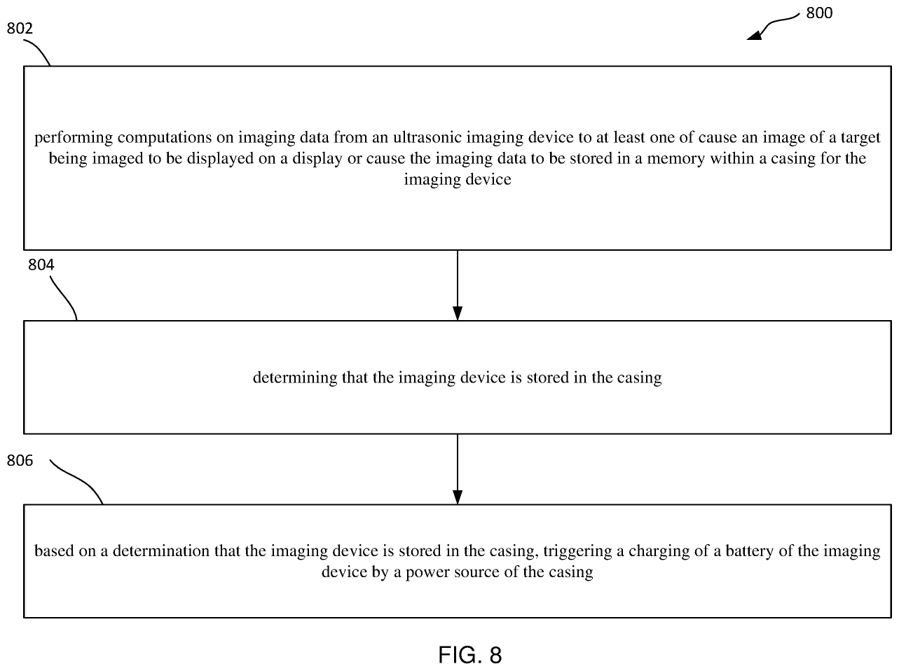

FIG. 8 is a flow chart of a process 800 to be performed at a casing for an ultrasonic imaging device according to some embodiments. At operation 802, the process includes performing computations on imaging data from the ultrasonic imaging device to at least one of cause an image of a target being imaged to be displayed on a display or cause the imaging data to be stored in a memory within the casing. At operation 804, the process includes determining that the imaging device is stored in the casing. At operation 806, the process includes, based on a determination that the imaging device is stored in the casing, triggering a charging of a battery of the imaging device by a power source of the casing

In an example, instructions implemented by processor 406 may be provided via the memory 404, or the processor 406 may be embodied as a non-transitory, machine-readable medium including code to direct the processor 406 to perform electronic operations in the casing. The processor 406 may access the non-transitory, machine-readable medium over the an interconnect between memory 404 and processor 406. For instance, the non-transitory, machine-readable medium may be embodied by memory 404 or a separate memory within processor 406, or may include specific storage units such as optical disks, flash drives, or any number of other hardware devices that may be plugged into the casing. The non-transitory, machine-readable medium may include instructions to direct the processor 406 to perform a specific sequence or flow of actions, for example, as described with respect to the flowchart(s) and block diagram(s) of operations and functionality depicted herein. As used herein, the terms “machine-readable medium” and “computer-readable medium” are interchangeable.

Any of the below-described Examples may be combined with any other example (or combination of examples), unless explicitly stated otherwise. Aspects described herein can also implement a hierarchical application of the scheme for example, by introducing a hierarchical prioritization of usage for different functions (e.g., low/medium/high priority, etc.),

Although implementations have been described with reference to specific exemplary aspects, it will be evident that various modifications and changes may be made to these aspects without departing from the broader scope of the present disclosure. Many of the arrangements and processes described herein can be used in combination or in parallel implementations. Accordingly, the specification and drawings are to be regarded in an illustrative rather than a restrictive sense. The accompanying drawings that form a part hereof show, by way of illustration, and not of limitation, specific aspects in which the subject matter may be practiced. The aspects illustrated are described in sufficient detail to enable those skilled in the art to practice the teachings disclosed herein. Other aspects may be utilized and derived therefrom, such that structural and logical substitutions and changes may be made without departing from the scope of this disclosure. This Detailed Description, therefore, is not to be taken in a limiting sense, and the scope of various aspects is defined only by the appended claims, along with the full range of equivalents to which such claims are entitled.

Such aspects of the inventive subject matter may be referred to herein, individually and/or collectively, merely for convenience and without intending to voluntarily limit the scope of this application to any single aspect or inventive concept if more than one is in fact disclosed.

While preferred embodiments of the present disclosure have been shown and described herein, it will be obvious to those skilled in the art that such embodiments are provided by way of example only. It is not intended that embodiments be limited by the specific examples provided within the specification. While embodiments of the disclosure have been described with reference to the aforementioned specification, the descriptions and illustrations of the embodiments herein are not meant to be construed in a limiting sense. Numerous variations, changes, and substitutions will now occur to those skilled in the art without departing from the concepts of the present disclosure. Furthermore, it shall be understood that all aspects of the various embodiments are not limited to the specific depictions, configurations, or relative proportions set forth herein, which depend upon a variety of conditions and variables. It should be understood that various alternatives to the embodiments described herein may be employed. It is therefore contemplated that the disclosure also covers any such alternatives, modifications, variations or equivalents.

### Examples

Illustrative examples of the technologies disclosed herein are provided below. An embodiment of the technologies may include any one or more, and any combination of, the examples described below.

Example 1 includes a casing to store a portable imaging device adapted to generate imaging data corresponding to a target being imaged using ultrasonic energy, the casing adapted to be opened and closed, and including: an exterior housing; an interior portion within the exterior housing to house the imaging device therein; a memory; a one or more processors coupled to the memory to perform computations on the imaging data from the imaging device to at least one of cause an image of the target to be displayed on a display or cause the imaging data to be stored in the memory; and a power source to supply charge to the imaging device.

Example 2 includes the subject matter of Example 1, wherein the memory, computing device and power source are in the interior portion of the casing, and the casing includes a base portion and a lid portion adapted to be fastened to one another when the casing is closed.

Example 3 includes the subject matter of Example 1, wherein the exterior housing is made of an impact-resistant material to protect the imaging device against impact when the imaging device is stored in the casing and when the casing is closed, and the interior portion includes a compliant material to provide padding for the imaging device when the imaging device is stored in the casing.

Example 4 includes the subject matter of Example 1, wherein the power source includes at least one of a battery or one or more solar panels, the casing further including charging contacts coupled to said at least one of the battery or the one or more solar panels, and positioned to be in contact with the imaging device to supply power to a battery of the imaging device.

Example 5 includes the subject matter of Example 4, wherein the solar panels are coupled to the battery to charge the battery.

Example 6 includes the subject matter of Example 3, wherein the compliant material defines a recess therein substantially conformal to portions of an exterior outline of the imaging device to retain the imaging device therein in a nested manner.

Example 7 includes the subject matter of Example 6, wherein the recess corresponds to a charging dock for the imaging device, and wherein the power source is coupled to the charging dock to charge the imaging device when nested within the recess.

Example 8 includes the subject matter of Example 6, further including a disinfecting mechanism positioned in the interior portion to cause disinfection of one or more portions of the imaging device when the imaging device is nested in the recess.

Example 9 includes the subject matter of Example 8, wherein the disinfecting mechanism includes one or more ultraviolet disinfecting light sources located at walls of the recess and adapted to direct disinfecting light toward the one or more portions of the imaging device.

Example 10 includes the subject matter of Example 9, wherein the one or more processors are to control an activation and deactivation of the light sources.

Example 11 includes the subject matter of Example 10, wherein the one or more processors are to activate the light sources in response to a determination that at least one of the imaging device is nested in the recess, the casing is closed.

Example 12 includes the subject matter of Example 1, further including wireless power circuitry including one or more charging coils positioned to be in alignment with one or more corresponding coils of the imaging device when the imaging device is stored in the casing to cause at least one of inductive or resonant charging of the imaging device.

Example 13 includes the subject matter of Example 1, wherein the one or more processors are to select an imaging mode for the imaging device from a plurality of selectable imaging modes, and to control the imaging device to operate based on a selected imaging mode, the selectable imaging modes including at least one of: a one-dimensional imaging mode, a two-dimensional imaging mode, a three-dimensional imaging mode, a Doppler imaging mode, a linear mode or a sector mode.

Example 14 includes the subject matter of Example 13, wherein the one or more processors are to select the imaging mode based on whether the imaging device or any portion thereof has exceeded one or more predetermined operating temperature thresholds.

Example 15 includes the subject matter of Example 1, wherein the one or more processors are to implement a feature identification algorithm to identify a target being imaged based on the image data, generate data based on an identification of the target, and cause communication of the data based on the identification of the target to at least one of a user of the imaging device or a remote device.

Example 16 includes the subject matter of Example 15, wherein the data based on an identification of the target corresponds to at least one of a marking on an image of the target, text communication, or voice communication.

Example 17 includes the subject matter of any one of Examples 15-16, wherein the data based on an identification of the target corresponds to guidance on a medical procedure to be performed on a patient, wherein the target is in the patient.

Example 18 includes the subject matter of any one of Examples 1-16, the casing configured for communication between the one or more processors and at least one of the display, the imaging device and a remote device by way of a wired or wireless connection, the communication including at least one of imaging data, metadata associated with the imaging data, voice data or text data.

Example 19 includes the subject matter of Example 18, wherein the metadata includes at least one of patient identifying information, date of the image data, or geographic location of image capture corresponding to the image data.

Example 20 includes the subject matter of Example 18, further including the display.

Example 21 includes the subject matter of Example 20, wherein the display includes a touchscreen.

Example 22 includes the subject matter of Example 21, wherein the one or more processors are to cause user selectable features to be displayed on the display such that a user may select one of the features using the touchscreen, the features including available commands from the user regarding ultrasonic imaging.

Example 23 includes the subject matter of Example 18, further including wireless communication circuitry to implement the communication, the wireless communication circuitry to cause wireless communication compliant with at least one of a Wi-Fi wireless communication protocol, a cellular wireless communication protocol, a mmWave wireless communication protocol, or a Bluetooth wireless communication protocol.

Example 24 includes the subject matter of Example 18, further including ports for wired connection to implement the communication, the ports to be compliant with at least one wired communication protocol including Ethernet, RS-232, RS-485, UART, USART, USB2, USB 3, USB 3.1, or USB-.C

Example 25 includes the subject matter of Example 18, the one or more processors to determine to cause the communication based on a storage capacity of the memory.

Example 26 includes the subject matter of Example 18, the one or more processors to determine at least one of an audio signal or a visual signal received from a remote device at the casing for a user of the imaging device based on imaging data sent to the remote device, and to at least one of cause the audio signal to be played back to the user on a speaker coupled to the one or more processors, or cause the visual signal to be displayed to the user on a display coupled to the one or more processors.

Example 27 includes the subject matter of Example 26, further including at least one of the speaker or the display.

Example 28 includes the subject matter of Example 18, wherein the processors are to, in response to a determination that the casing is closed, trigger an wired or wireless upload of data, including imaging data, to another device from one of a memory of the imaging device if the imaging device is stored in the casing, or the memory of the casing, wherein the other device is one of a device local to the casing or a remote device.

Example 29 includes the subject matter of Example 28, the one or more processors to, in response to a determination that the data has been uploaded to said another device, cause the data to be erased from said one of the memory of the imaging device or the memory of the casing.

Example 30 includes the subject matter of any one of Examples 1-16, wherein the one or more processors are to determine an audio signal from a microphone coupled thereto, and to cause communication of data corresponding to the audio signal to a remote device.

Example 31 includes the subject matter of Example 28, further including the microphone.

Example 32 includes the subject matter of any one of Examples 1-16, wherein the one or more processors are to cause imaging data to be read from the memory, and to be communicated to at least one of a user of the imaging device, the display, or a remote device.

Example 33 includes the subject matter of any one of Examples 1-16, further including an electrocardiogram (ECG) subsystem including circuitry to at least one of receive and process electrode signals from ECG electrodes.

Example 34 includes the subject matter of Example 31, wherein the one or more processors including the ECG subsystem.

Example 35 includes the subject matter of any one of Examples 1-16, wherein the one or more processors are to cause the imaging data to be cryptographically encoded prior to causing the imaging data to be written into the memory.

Example 36 includes the subject matter of any one of Examples 1-16, further including a lock, the lock including at least one of a mechanical fastening device or an electronic fastening device to be released by at least one of a password or a physical key including a keycard, a fingerprint, a radio frequency identification (RFID) card, or a security token.

Example 37 includes the subject matter of any one of Examples 1-16, further including one or more compartments to store accessories of the imaging device.

Example 38 includes the subject matter of any one of Examples 1-16, further including one or more power charging ports coupled to the power source, and one or more data communication ports coupled to the one or more processors.

Example 39 includes the subject matter of Example 38, wherein the one or more power charging ports and the one or more data communication ports are configured to couple the casing to an external docking station.

Example 40 includes the subject matter of any one of Examples 1-16, further including charge level indicators to indicate at least a charge level of the power source or a charge level of a battery of the imaging device when the imaging device is stored in the casing.

Example 41 includes a set including the casing of any one of Examples 1-16, and the imaging device.

Example 42 includes a method to be performed at a casing adapted to store a portable imaging device configured to generate imaging data corresponding to a target being imaged using ultrasonic energy, the casing adapted to be opened and closed and including a memory, the method including: performing computations on the imaging data from the imaging device to at least one of cause an image of the target to be displayed on a display or cause the imaging data to be stored in the memory; determining that the imaging device is stored in the casing; and based on a determination that the imaging device is stored in the casing, triggering a charging of a battery of the imaging device by a power source of the casing.

Example 43 includes the subject matter of Example 42, further including triggering a disinfecting mechanism to cause disinfection of one or more portions of the imaging device based on at least one of a determination that the imaging device is stored in the casing.

Example 44 includes the subject matter of Example 43, wherein the disinfecting mechanism includes one or more ultraviolet disinfecting light sources located in the casing and adapted to direct disinfecting light toward the one or more portions of the imaging device.

Example 45 includes the subject matter of Example 44, further including controlling an activation and deactivation of the light sources.

Example 46 includes the subject matter of Example 44, further including activating the light sources when the casing is closed, and deactivating the light sources when the casing is open.

Example 47 includes the subject matter of Example 42, further including triggering a charging of a battery of the casing by the power source.

Example 48 includes the subject matter of Example 47, wherein the power source includes solar panels, the method further including activating the solar panels to charge at least one of the battery of the casing or the battery of the imaging device.

Example 49 includes the subject matter of Example 48, further including activating the solar panels in response to a determination that the casing is closed.

Example 50 includes the subject matter of Example 42, wherein triggering the charging includes triggering a wireless power circuitry including one or more charging coils of the casing positioned to be in alignment with one or more corresponding coils of the imaging device when the imaging device is stored in the casing to cause at least one of inductive or resonant charging of the imaging device.

Example 51 includes the subject matter of Example 42, further including selecting an imaging mode for the imaging device from a plurality of selectable imaging modes, and controlling the imaging device to operate based on a selected imaging mode, the selectable imaging modes including at least one of: a one-dimensional imaging mode, a two-dimensional imaging mode, a three-dimensional imaging mode, a Doppler imaging mode, a linear mode or a sector mode.

Example 52 includes the subject matter of Example 51, wherein selecting the imaging mode is based on whether the imaging device or any portion thereof has exceeded one or more predetermined operating temperature thresholds.

Example 53 includes the subject matter of Example 42, further including implementing a feature identification algorithm to identify a target being imaged based on the image data, generating data based on an identification of the target, and causing communication of the data based on the identification of the target to at least one of a user of the imaging device or a remote device.

Example 54 includes the subject matter of Example 53, wherein the data based on an identification of the target corresponds to at least one of a marking on an image of the target, text communication, or voice communication.

Example 55 includes the subject matter of Example 52, wherein the data based on an identification of the target corresponds to guidance on a medical procedure to be performed on a patient, wherein the target is in the patient.

Example 56 includes the subject matter of Example 52, further including communicating between the casing and at least one of the display, the imaging device and a remote device by way of a wired or wireless connection, the communicating including transmitting or receiving at least one of imaging data, metadata associated with the imaging data, voice data or text data.

Example 57 includes the subject matter of Example 56, wherein the metadata includes at least one of patient identifying information, date of the image data, or geographic location of image capture corresponding to the image data.

Example 58 includes the subject matter of Example 56, further including causing user selectable features to be displayed on the display such that a user may select one of the features using a touchscreen feature of the display, the features including available commands from the user regarding ultrasonic imaging.

Example 59 includes the subject matter of Example 56, wherein communicating includes using a wireless communication circuitry of the casing, the wireless communication circuitry to cause wireless communication compliant with at least one of a Wi-Fi wireless communication protocol, a cellular wireless communication protocol, a mmWave wireless communication protocol, or a Bluetooth wireless communication protocol.

Example 60 includes the subject matter of Example 56, wherein communicating includes using ports of the casing for wired connection, the ports to be compliant with at least one wired communication protocol including Ethernet, RS-232, RS-485, UART, USART, USB2, USB 3, USB 3.1, or USB-.C

Example 61 includes the subject matter of Example 56, wherein communicating is based on a storage capacity of the memory.

Example 62 includes the subject matter of Example 56, further including determining at least one of an audio signal or a visual signal received from a remote device at the casing for a user of the imaging device based on imaging data sent to the remote device, and at least one of causing the audio signal to be played back to the user on a speaker, or causing the visual signal to be displayed on the display.

Example 63 includes the subject matter of Example 56, further including, in response to a determination that the casing is closed, triggering a wired or wireless upload of data, including imaging data, to another device from one of a memory of the imaging device if the imaging device is stored in the casing, or the memory of the casing, wherein the other device is one of a device local to the casing or a remote device.

Example 64 includes the subject matter of Example 63, further including, in response to a determination that the data has been uploaded to said another device, causing the data to be erased from said one of the memory of the imaging device or the memory of the casing.

Example 65 includes the subject matter of Example 42, further including determining an audio signal from a microphone coupled to the casing, and causing communication of data corresponding to the audio signal to a remote device.

Example 66 includes the subject matter of Example 42, further including causing imaging data to be read from the memory, and to be communicated to at least one of a user of the imaging device, the display, or a remote device.

Example 67 includes the subject matter of Example 42, further including at least one of receiving and processing electrode signals from ECG electrodes of an electrocardiogram (ECG) subsystem of the casing.

Example 68 includes the subject matter of Example 42, further including causing the imaging data to be cryptographically encoded prior to causing the imaging data to be written into the memory.

Example 71 includes one or more computer-readable media comprising a plurality of instructions stored thereon that, when executed, cause one or more processors to perform the method of any one of Examples 42-68.

Example 70 includes an apparatus including means for performing a method of any one of claims 42-68.

Example 71 includes a product comprising one or more tangible computer-readable non-transitory storage media comprising computer-executable instructions operable to, when executed by at least one computer processor, enable the at least one processor to perform the method of any one of Examples 42-68.

1-70. (canceled)

71. A casing to store a portable imaging device adapted to generate imaging data corresponding to a target being imaged using ultrasonic energy, the casing adapted to be opened and closed, and including:
an exterior housing;
an interior portion within the exterior housing to house the imaging device therein;
a memory;
a one or more processors coupled to the memory to perform computations on the imaging data from the imaging device to at least one of cause an image of the target to be displayed on a display or cause the imaging data to be stored in the memory; and
a power source to supply charge to the imaging device.

72. The casing of claim 71, wherein the memory, computing device and power source are in the interior portion of the casing, and the casing includes a base portion and a lid portion adapted to be fastened to one another when the casing is closed.

73. The casing of claim 71, wherein the exterior housing is made of an impact-resistant material to protect the imaging device against impact when the imaging device is stored in the casing and when the casing is closed, and the interior portion includes a compliant material to provide padding for the imaging device when the imaging device is stored in the casing.

74. The casing of claim 73, wherein the compliant material defines a recess therein substantially conformal to portions of an exterior outline of the imaging device to retain the imaging device therein in a nested manner.

75. The casing of claim 71, wherein the power source includes at least one of a battery or one or more solar panels, the casing further including charging contacts coupled to said at least one of the battery or the one or more solar panels, and positioned to be in contact with the imaging device to supply power to a battery of the imaging device.

76. The casing of claim 75, wherein the recess corresponds to a charging dock for the imaging device, and wherein the power source is coupled to the charging dock to charge the imaging device when nested within the recess.

77. The casing of claim 75, further including a disinfecting mechanism positioned in the interior portion to cause disinfection of one or more portions of the imaging device when the imaging device is nested in the recess.

78. The casing of claim 77, wherein the disinfecting mechanism includes one or more ultraviolet disinfecting light sources located at walls of the recess and adapted to direct disinfecting light toward the one or more portions of the imaging device.

79. The casing of claim 71, further including wireless power circuitry including one or more charging coils positioned to be in alignment with one or more corresponding coils of the imaging device when the imaging device is stored in the casing to cause at least one of inductive or resonant charging of the imaging device.

80. The casing of claim 71, wherein the one or more processors are to select an imaging mode for the imaging device from a plurality of selectable imaging modes, and to control the imaging device to operate based on a selected imaging mode, the selectable imaging modes including at least one of: a one-dimensional imaging mode, a two-dimensional imaging mode, a three-dimensional imaging mode, a Doppler imaging mode, a linear mode or a sector mode.

81. The casing of claim 80, wherein the one or more processors are to select the imaging mode based on whether the imaging device or any portion thereof has exceeded one or more predetermined operating temperature thresholds.

82. The casing of claim 71 wherein the one or more processors are to implement a feature identification algorithm to identify a target being imaged based on the image data, generate data based on an identification of the target, and cause communication of the data based on the identification of the target to at least one of a user of the imaging device or a remote device.

83. The casing of claim 82, wherein the data based on an identification of the target corresponds to at least one of a marking on an image of the target, text communication, or voice communication.

84. The casing of claim 82, wherein the data based on an identification of the target corresponds to guidance on a medical procedure to be performed on a patient, wherein the target is in the patient.

85. The casing of claim 82, the casing configured for communication between the one or more processors and at least one of the display, the imaging device and a remote device by way of a wired or wireless connection, the communication including at least one of imaging data, metadata associated with the imaging data, voice data or text data.

86. The casing of claim 71, further including the display.

87. A system comprising:
a portable imaging device adapted to generate imaging data corresponding to a target being imaged using ultrasonic energy; and
a casing to house the imaging device, the casing comprising:
a dock to house the imaging device therein;
a memory;
one or more processors coupled to the memory to perform computations on the imaging data from the imaging device to at least one of cause an image of the target to be displayed on a display or cause the imaging data to be stored in the memory; and
a power source to supply charge to the imaging device.

88. The system of claim 87, wherein the power source includes at least one of a battery or one or more solar panels, the casing further including charging contacts coupled to said at least one of the battery or the one or more solar panels, and positioned to be in contact with the imaging device to supply power to a battery of the imaging device.

89. The system of claim 87, further including a disinfecting mechanism positioned to cause disinfection of one or more portions of the imaging device when the imaging device is housed in the casing.

90. The system of claim 87, further including the display.

91. A method to be performed at a casing adapted to store a portable imaging device configured to generate imaging data corresponding to a target being imaged using ultrasonic energy, the casing adapted to be opened and closed and including a memory, the method including:
performing computations on the imaging data from the imaging device to at least one of cause an image of the target to be displayed on a display or cause the imaging data to be stored in the memory;
determining that the imaging device is stored in the casing; and
based on a determination that the imaging device is stored in the casing, triggering a charging of a battery of the imaging device by a power source of the casing.

92. The method of claim 91, further including triggering a disinfecting mechanism to cause disinfection of one or more portions of the imaging device based on at least one of a determination that the imaging device is stored in the casing.

93. The method of claim 91, further including selecting an imaging mode for the imaging device from a plurality of selectable imaging modes, and controlling the imaging device to operate based on a selected imaging mode, the selectable imaging modes including at least one of: a one-dimensional imaging mode, a two-dimensional imaging mode, a three-dimensional imaging mode, a Doppler imaging mode, a linear mode or a sector mode.

94. The method of claim 91, further including implementing a feature identification algorithm to identify a target being imaged based on the image data, generating data based on an identification of the target, and causing communication of the data based on the identification of the target to at least one of a user of the imaging device or a remote device.

95. The method of claim 91, further including causing imaging data to be read from the memory, and to be communicated to at least one of a user of the imaging device, the display, or a remote device.
